(19) United States
(12) Patent Application Publication
(10) Pub. No.: US 2024/0335355 A1
(43) Pub. Date: Oct. 10, 2024

(54) VIAL ADAPTOR

(71) Applicant: Alveo Technologies, Inc., Alameda, CA (US)

(72) Inventors: Daniel Jordan Wade, Alameda, CA (US); Aman Adam Ullah, Alameda, CA (US)

(21) Appl. No.: 18/690,897

(22) PCT Filed: Sep. 23, 2022
(86) PCT No.: PCT/US2022/044516

§ 371 Date: Mar. 11, 2024

Related U.S. Application Data
(60) Provisional application No. 63/248,301, filed on Sep. 24, 2021.

(51) Int. Cl. A61J 1/20

(52) U.S. Cl. CPC A61J 1/2096

(57) ABSTRACT

A vial adaptor is disclosed. The vial adaptor can include a cap member and a piston. The cap member can removably couple to a vial storing, for example, liquid constituent for facilitating sample collection for detecting pathogens. The piston can distally translate through the cap member and into the vial to generate volume displacement and positive pressure, which can cause the liquid constituent stored in the vial to flow out from the vial via a channel formed within the piston. The piston can include a cover that creates a water-tight seal with an inner surface of the vial.

### PRIORITY AND CROSS-REFERENCE TO RELATED APPLICATIONS

This application claims the benefit of U.S. Provisional Application 63/248,301, filed on Sep. 24, 2021, which is hereby incorporated by reference in its entirety.

### FIELD

The present application is generally related to a vial adaptor for transferring materials, such as a fluid containing a biological sample, from a vial to another device such as, a cartridge for sensing and/or identifying pathogens, genomic materials, proteins, and/or other small molecules or biomarkers.

### BACKGROUND

Pathogens may be identified by detecting genomic material (DNA or RNA) in a biological sample. In conventional nucleic acid testing (“NAAT”), DNA in a biological sample is exponentially copied using a molecular amplification processes such as, the polymerase chain reaction (“PCR”) or Loop-Mediated Isothermal Amplification (“LAMP”) until the quantity of DNA present is great enough to be measurable. In the case of RNA, the genomic material of many viruses, an additional step can be included to first transcribe the RNA into DNA before amplification. Liquid constituents such as amplification reagents, buffer solutions, water, mucin mitigating agents, or other desired liquid constituents are typically used in the testing processes, some of which assist in extracting the DNA, RNA or both from a target agent present in the biological sample such as, mold, fungus, bacteria, virus, or a microbe. These liquid constituents need to be efficiently introduced into devices that facilitate NAAT, especially in situations where pipettes are not readily available. There is a lasting need for improved pathogen detection methods and tools, which facilitate the transfer of biological samples and liquid constituents into devices that are used for NAAT.

### SUMMARY

According to one aspect of the disclosure, a vial adaptor that can facilitate transfer of fluid from a vial to a receiving device is disclosed. The vial adaptor can include a cap member comprising a proximal opening, a distal opening, and a threaded portion that can removably couple with a corresponding threaded portion formed on an opening of a vial; a piston including a cover, a distal end, a proximal end, a channel, and a body extending between the distal end and the proximal end and comprising a head, the cover can surround the head and abut an inner surface of the vial, the channel extending between the distal end and the proximal end of the piston and configured to allow flow of fluid through the piston between the distal end and the proximal end; a ratchet system including: a first arm formed about the proximal opening of the cap member, at least a portion of the first arm extending inward towards a center of the proximal opening of the cap member, the first arm including a tip; and a first rack formed along the body of the piston, the first rack can engage the first arm, wherein the engagement between the first rack and the first arm allows the piston to translate in a first direction, and wherein a translation of the piston in the first direction can cause a positive pressure change in the vial and withdrawal of fluid stored in the vial through the channel of the piston.

In some embodiments, the first direction can be a direction towards the vial.

In some embodiments, the engagement between the first rack and the first arm can prevent the piston from translating in a second direction.

In some embodiments, the second direction can be a direction away from the vial.

In some embodiments, the channel can include a proximal aperture and a distal aperture, the channel extending between the proximal aperture and the distal aperture, the distal aperture formed on a distal portion of the head of the body of the piston, the proximal aperture formed about the proximal end of the piston. The cover can include an opening that aligns with the proximal aperture of the channel, and wherein the fluid stored in the vial flows into the piston via the opening of the cover and the proximal aperture of the channel.

In some embodiments, an outer circumference of the cover can create a seal with the inner surface vial. The cover can be a molded elastomer.

In some embodiments, the first rack includes a plurality of tooth, and wherein each of the plurality of tooth includes a leading edge and a stopping edge. The leading edges of the first rack facilitates movement of the first arm, and wherein the stopping edges of the first rack prevents movement of the first arm. The first arm slides along the leading edges of the first rack when the piston is translated in the first direction, and wherein the first arm abuts against one of the stopping edges of the first rack when the piston is translated in the second direction.

In some embodiments, the proximal end of the piston removably can couple with a receiving device.

In some embodiments, the piston can include a stopper extending radially and circumferentially outwards from the body of the piston, wherein the stopper can abut the cap member when the piston is in its distal-most position with respect to the cap member.

In some embodiments, a width of the channel can increase from the distal end of the piston to the proximal end of the piston.

In some embodiments, the tip of the first arm can be curved inwards towards an axis orthogonal to the proximal opening and the distal opening.

In some embodiments, the vial adaptor can include: a second arm; and a second rack formed along the body of the piston and configured to engage the second arm. The second arm can be positioned opposite of the first arm, and wherein the second rack can be formed on an opposite side of the body of the piston.

According to another aspect of the disclosure, a vial adaptor is disclosed. The vial adaptor can include: a cap member that can removably coupled with an opening of a vial; and a piston including a distal end, a proximal end, a body extending between the distal end and the proximal end, and a channel, the channel can allow flow of fluid through the piston between a distal aperture formed on the distal end of the piston and a proximal aperture formed on the proximal end of the piston, the piston can translate distally through the cap member and towards the vial, wherein a distal translation of the piston can cause a positive pressure change in the vial and withdrawal of fluid stored in the vial through the channel of the piston.

In some embodiments, the cap member can include an arm, wherein the piston can include a rack, and wherein the arm can engage the rack to allow the piston to translate distally through the cap member towards the vial and prevent the piston to translate proximally through the cap member away from the vial. The arm can include a tip, and wherein the tip can be curved inwards towards an axis parallel to the body of the piston. The rack can include a plurality of tooth, wherein each of the plurality of tooth can include a leading edge and a stopping edge, and wherein the arm can slide along the leading edges of the plurality of tooth and the piston can be translated distally and abut one of the stopping edges of the plurality of tooth to prevent the piston from translating proximally.

In some embodiments, the proximal end of the piston can be removably coupled with a receiving device.

In some embodiments, the piston can include a stopper extending radially and circumferentially outwards from the body of the piston, wherein the stopper can abut the cap member when the piston is in its distal-most position with respect to the cap member.

In some embodiments, a width of the channel can increase from the distal aperture to the proximal aperture.

In some embodiments, the tip of the first arm can be curved inwards towards an axis orthogonal to the proximal opening and the distal opening.

In some embodiments, the vial adaptor can include a cover, wherein the cover can cover the distal end of the body of the piston. The cover can be a molded elastomer. The cover can abut an inner surface of the vial to create a seal between the cover and the inner surface of the vial.

According to another aspect of the disclosure, a piston for withdrawing fluid stored in a vial is disclosed. The piston can include: a distal end; a proximal end; a body extending between the distal end and the proximal end, the body including a head including a distal aperture, the proximal end including a proximal aperture; a channel formed within the body and extending between the distal aperture and the proximal aperture; the head of the piston that can move towards and into a vial and generate a positive pressure change in the vial, thereby causing a flow of fluid stored in the vial into the channel via the distal aperture and out of the channel via the proximal aperture.

In some embodiments, a width of the channel increases from the distal aperture to the proximal aperture.

In some embodiments, the piston can include a rack comprising a plurality of tooth. The rack can be formed along a length of the body of the piston. Each of the plurality of tooth can include a leading edge and a stopping edge. The leading edges of the plurality of tooth can allow an arm of a cap member to slide along in a first direction, and wherein the stopping edges of the plurality of tooth can prevent the arm of the cap member from moving in a second direction opposite from the first direction.

In some embodiments, the piston can move through a proximal opening and a distal opening of a cap member, and wherein the cap member can be attached to an opening of the vial.

In some embodiments, the cap member can be positioned between the piston and the vial.

In some embodiments, the piston can include a cover. The cover can cover the distal end of the piston, and wherein an outer circumference of the cover can abut an inner surface of the vial to create a seal between the cover and the vial. The cover can be a molded elastomer.

In some embodiments, the proximal end of the piston can removably couple with a receiving device.

According to another aspect of the disclosure, a method of withdrawing fluid stored in a vial via a vial adaptor is disclosed. The method can include: coupling a distal opening of a cap member of a vial adaptor to an opening of a vial, the cap member including a proximal opening opposite of the distal opening; coupling a proximal end of a piston of the vial adaptor to an opening of a receiving device; distally translating the piston towards the vial and through the proximal opening and the distal opening of the cap member, a head of the piston can abut an inner surface of the vial to create a water-tight seal between the head of the piston and the inner surface of the vial, a distal translation of the piston towards the vial can generate positive pressure change in the vial and cause a flow of fluid stored in the vial.

In some embodiments, the head of the piston can be a molded elastomer.

In some embodiments, the cap member can include an arm and the piston can include a rack having a plurality of tooth, and wherein the arm of the cap member and the plurality of tooth of the piston can engage during the distal translation of the piston.

In some embodiments, each of the plurality of tooth of the rack can include a leading edge and a stopping edge, wherein the arm slides along the leading edges of the plurality of tooth of the rack during the distal translation of the piston, and wherein the stopping edges of the plurality of tooth of the rack can prevent a proximal translation of the piston away from the vial.

In some embodiments, the piston can include a channel extending between a distal aperture formed about a distal end of the piston and a proximal aperture formed about the proximal end of the piston.

In some embodiments, the piston can include a stopper. The stopper can abut the cap member when the piston is in its distal-most position. The stopper can abut the receiving device when the proximal end of the piston is coupled to the opening of the receiving device.

### BRIEF DESCRIPTION OF THE DRAWINGS

FIGS. 1A and 1B illustrate various views of an example vial adaptor coupled to a vial.

FIG. 2 illustrates an exploded view of the vial adaptor of FIGS. 1A and 1B, showing various components of the vial adaptor.

FIG. 3 illustrates a perspective view of the vial adaptor of FIGS. 1A and 1B.

FIG. 4A illustrates a perspective view of a piston portion of the vial adaptor of FIGS. 1A and 1B without a cap portion.

FIG. 4B illustrates the piston portion of FIG. 4A showing a tip separated from a piston body.

FIG. 5A illustrates a cross-sectional view of the syringe portion of FIG. 4A along the lines 5A-5A.

FIG. 5B illustrates a cross-sectional view of the syringe portion of FIG. 4B along the lines 5B-5B.

FIGS. 6A and 6B illustrate various views of a cap portion of the vial adaptor of FIGS. 1A and 1B.

FIG. 7A illustrates a cross-sectional view of the vial adaptor of FIGS. 1A and 1B prior to actuation of a piston of the vial adaptor.

FIG. 7B illustrates a cross-sectional view of the vial adaptor of FIGS. 1A and 1B during actuation of a piston of the vial adaptor.

FIG. 7C illustrates a cross-sectional view of the vial adaptor of FIGS. 1A and 1B showing a piston of the vial adaptor in its distal-most position.

FIG. 8A illustrates the vial adaptor of FIGS. 1A and 1B and a receiving device.

FIG. 8B illustrates the vial adaptor of FIGS. 1A and 1B coupled to a receiving device.

FIG. 9 illustrates an example method of transferring fluid from a vial to a receiving device using the vial adaptor of FIGS. 1A and 1B.

### DETAILED DESCRIPTION

Aspects of the disclosure herein concern an adaptor that facilitates the flow of liquid constituents from a standard vial to a target sensing device or a diagnostic device e.g., a cartridge configured to detect a pathogen. The adaptor may be coupled to a standard vial and a standard target sensing device without any modification to the vial or the target sensing device. In some aspects, the adaptor is configured to inhibit or prevent re-aspiration of the liquid constituent into the vial.

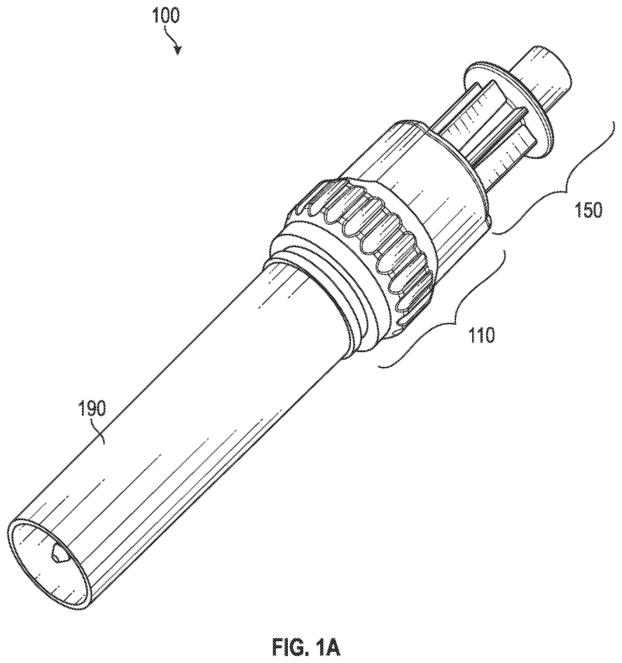

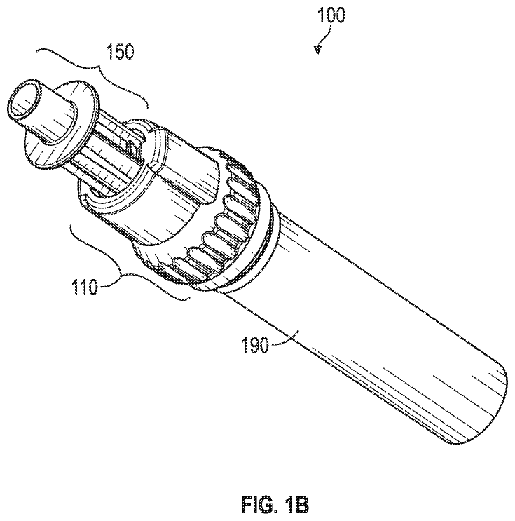

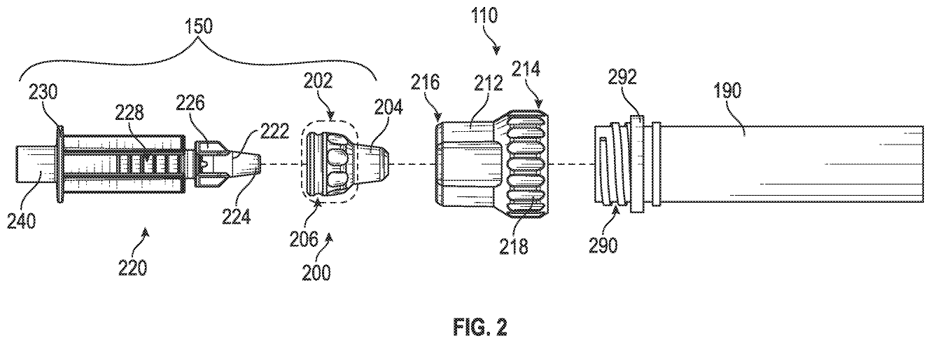

FIGS. 1A and 1B illustrate various views of an example vial adaptor 100. The vial 190 can be, in some embodiments, an off-the-shelf vial or the vial can be specifically or custom manufactured. The adaptor 100 can include a cap member 110 and a piston 150. The cap member 110 can be removably coupled to a vial 190 during use and can be decoupled after use. For example, the vial 190 can include a threaded or interlocking portion 290 (an example is shown in FIG. 2), which corresponds to a threaded or interlocking portion 620 of the cap member 110. The piston 150 can be slidingly inserted into a cavity formed by the cap member 110 and translate towards the vial 190 through the cap member 110. In some embodiments, the cap member 110 can include a locking mechanism that allows the piston 150 to only translate distally (for example, towards the vial 190) and not proximally (for example, away from the vial 190). The movement of the piston 150 (for example, distal translation through the cap member 110 towards the vial 190) can generate a positive pressure and cause fluid stored in the vial 190 to flow out of the vial 190 through the vial adaptor 100.

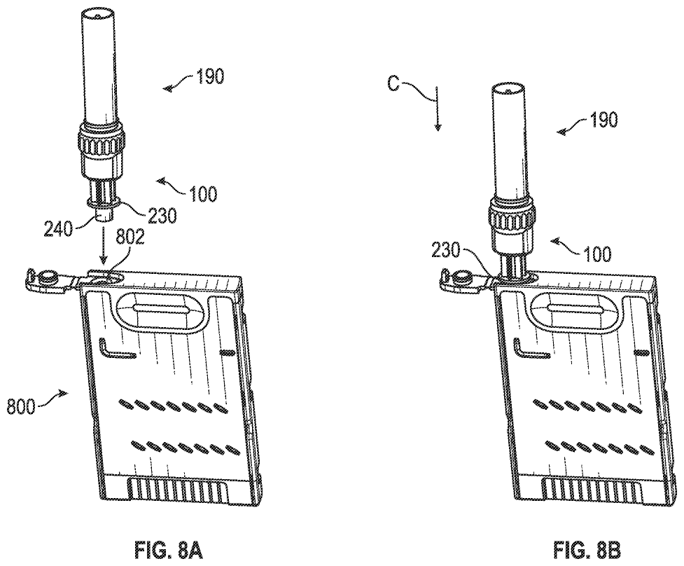

The piston 150 can include a proximal end (for example, an end opposite the vial 190 as shown in FIG. 1A) that can removably couple with an opening of a target sensing device or a diagnostic device (for example, see FIGS. 8A and 8B).

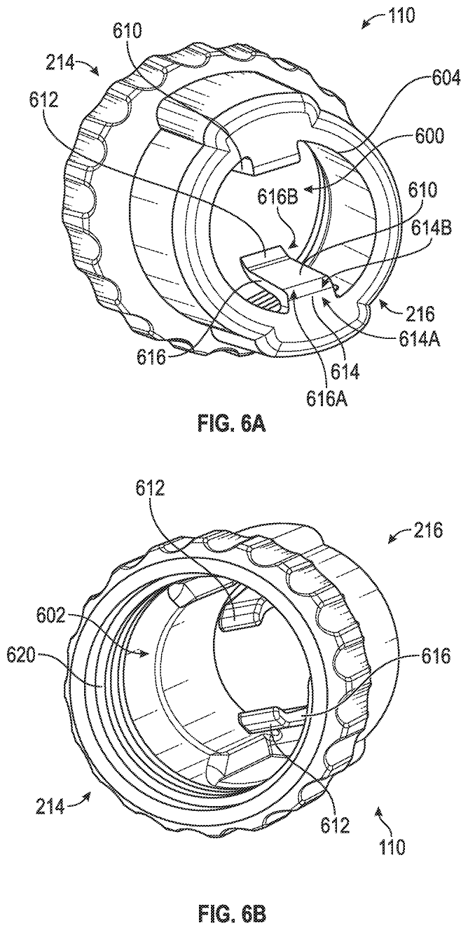

FIG. 2 illustrates an exploded view of the vial adaptor 100 and the vial 190. The cap member 110 can include a distal end 214 (for example, an end that is proximate to the vial 190) and a proximal end 216 (for example, an end that is distal from the vial 190). The proximal end 216 can include an opening dimensioned to receive the piston 150. The distal end 214 can include an opening dimensioned to couple with the vial 190. For example, the distal end 214 of the cap member 110 can include an inner surface having the threaded portion 620 (see FIG. 6B). Additionally, the distal end 214 of the cap member 110 can extend over at least a portion of the threaded portion 290 of the vial 190 when coupled to the vial 190. The coupling between the vial 190 and the cap member 110 can be leak-proof to prevent or inhibit fluid stored in the vial 190 from leaking during the transfer process.

The vial 190 can include the threaded portion 290 and a protrusion 292. The protrusion 292 can engage the distal end 214 of the cap member 110 and stop further distal translation of the cap member 110.

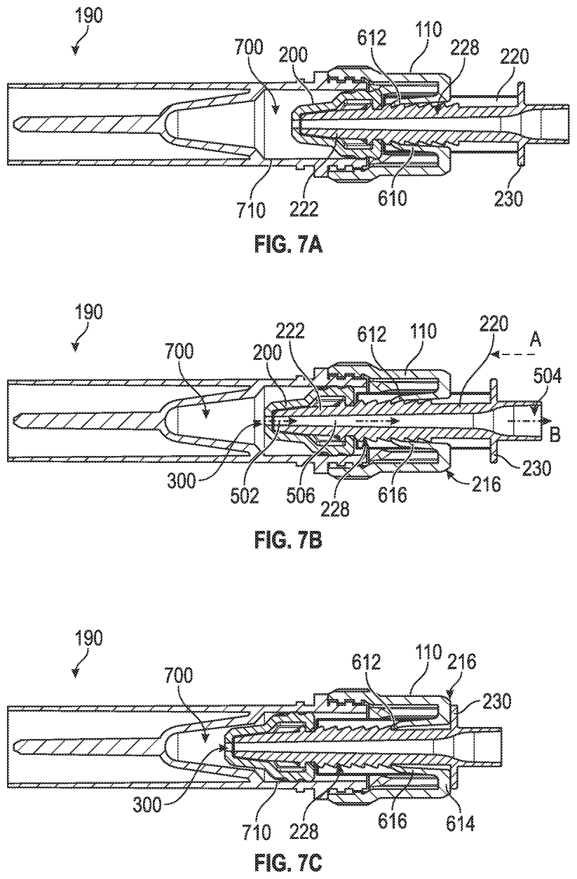

The piston 150 can include a cover 200 and a body 220. The cover 200 and the piston body 220 can be removably coupled. The cover 200 can include a base 202 and a tip 204. The base 202 that can be dimensioned such that its outer circumference 206 can abut against an inner surface (for example, inner surface 710 shown in FIG. 7A) of the vial 190 to create a seal. The seal between the inner surface 710 of the vial 190 and the outer circumference 206 of the base 202 can be maintained while the piston 150 is distally translated through the cap member 110 towards the vial 190. Additionally, the seal between the inner surface 710 of the vial 190 and the outer circumference 206 of the base 202 can prevent or inhibit fluid stored in the vial 190 from leaking between the base 202 of the cover 200 and the inner surface 710 of the vial 190 during use. Additionally, the seal between the inner surface 710 of the vial 190 and the outer circumference 206 of the base 202 can generate a positive pressure gradient or volume displacement in the vial 190, which can cause fluid stored in the vial 190 to flow out of the vial 190 via the vial adaptor 100.

In some embodiments, the cover 200 is made from a material that can generate desired amount of friction against the inner surface 710 of the vial 190 (for example, inner surface 710 shown in FIG. 7A). The friction between the cover 200 and the inner surface 710 of the vial 190 can prevent or inhibit accidental distal translation of the piston 150. In some embodiments, the cover 200 is a molded elastomer.

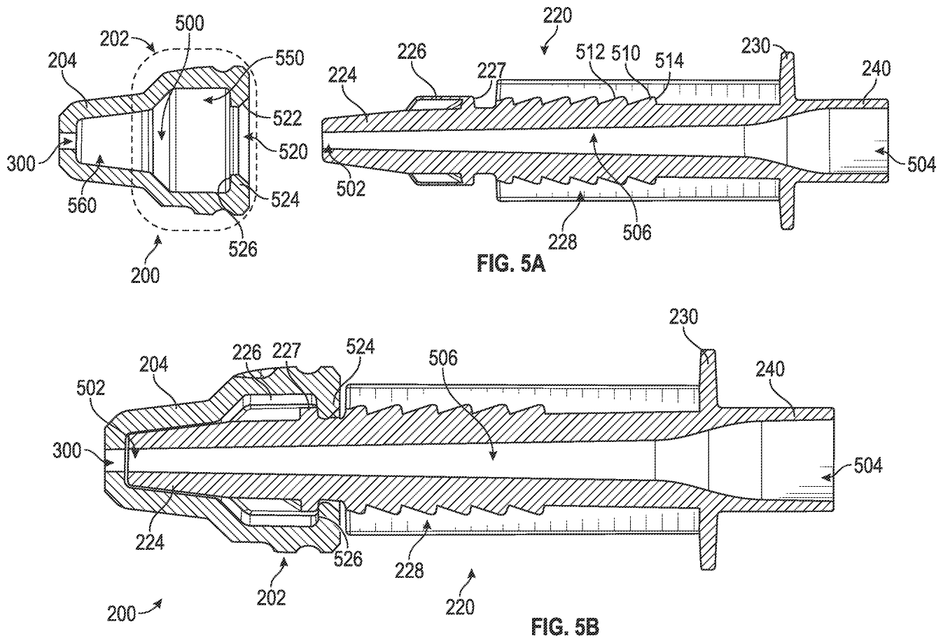

The body 220 of the piston 150 can include a head 222, one or more sets of racks 228, a stopper 230, and a proximal portion 240. The head 222 can include one or more flaps 226 and a distal end 224. The head 222 can be positioned inside the cover 200 (for example, in a cavity 500 as shown in FIG. 5A) when the head 222 is coupled with the cover 200. The flaps 226 can each include a rear edge 227 that can abut against an inner surface of the cover 200 and prevent the cover 200 from slipping off of the head 222. The distal end 224 can be shaped such that when the head 222 is inserted into the cavity 500 of the cover 200, the tip 204 and its inner surface wraps about an outer surface of the distal end 224 (for example, as shown in FIG. 5B). This can provide a desired seal between the cover 200 and the head 222 (for example, an inner surface of the distal end 204 and an outer surface of the distal end 224) that can prevent or inhibit flow of, for example, fluid stored in the vial 190 into an area between the cover 200 and the head 222 during use.

The racks 228 can be formed on the body 220. For example, the racks 228 can be formed along a longitudinal axis defined by the length of the body 220 (as shown in FIGS. 5A and 5B). The racks 228 can include teeth 510 that can engage corresponding arms (for example, arms 610 shown in FIGS. 6A and 6B) of the cap member 110 to restrict the movement of the piston 150 through the cap member 110. The racks 228 and the corresponding arms (for example, the arms 610) of the cap member 110 can be a locking mechanism that restricts the movement of the piston 150 through the cap member 110.

The stopper 230 can extend radially outward from an outer circumference of the body 220 proximate to the proximal portion 240. The stopper 230 can engage the proximal end 216 of the cap member 110 to prevent further movement (for example, distal translation) of the piston 150 with respect to the cap member 110 (for example, through the cap member 110). Additionally, the stopper 230 can abut against an opening of a testing device (e.g., a cartridge for sensing and/or identifying pathogens, genomic materials, proteins, and/or other small molecules or biomarkers) or another storage device (for example, for fluid samples) during use such that fluid stored in the vial 190 can be transferred to the testing device or the other storage device via the vial adaptor 100.

In some embodiments, the piston 150 can only translate distally (for example, towards the vial 190) and cannot translate proximally (for example, away from the vial 190). The movement of the piston 150 with respect to the cap member 110 is further described herein.

In some embodiments, the piston 150 and the cap member 110 may be integrated into a single device, while the piston 150 being able to move (for example, translate distally or proximally) with respect to the cap member 110.

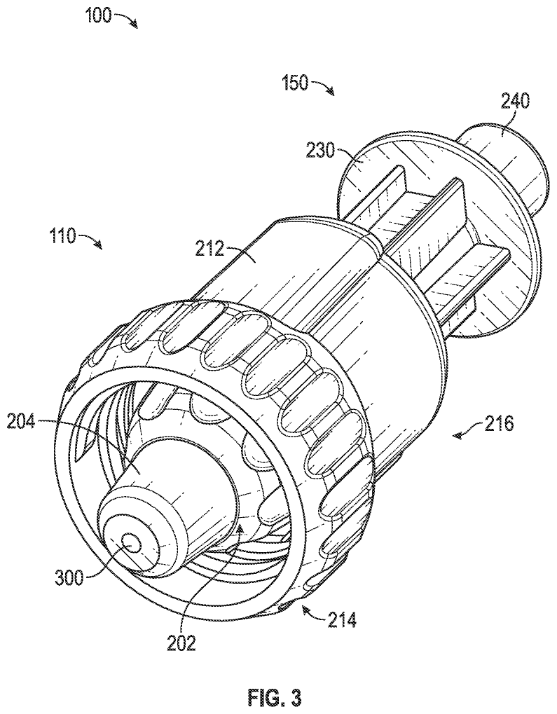

FIG. 3 illustrates a perspective view of the vial adaptor 100. The cover 200 can include a distal aperture 300 formed on a distal end of the tip 204. During use, as the piston 150 distally translates towards the vial 190, the cover 200 and the distal aperture 300 can move, for example, into a cavity 700 (see FIGS. 7A-7C) of the vial 190. As the cover 200 moves into the cavity (for example, the cavity 700) of the vial 190, it displaces the volume of the cavity and generates a positive pressure change in the vial 190. Because of the seal between the cover 200 and the inner surface (for example, the inner surface 710) of the vial 190, the volume displacement generated by the distal movement of the piston 150 (and the cover 200) can cause flow of fluid stored in the vial 190 via the distal aperture 300 and out of the vial 190 via the vial adaptor 100. In some embodiments, at least a portion of the cover 200 and the distal aperture 300 extends into a portion of the vial 190.

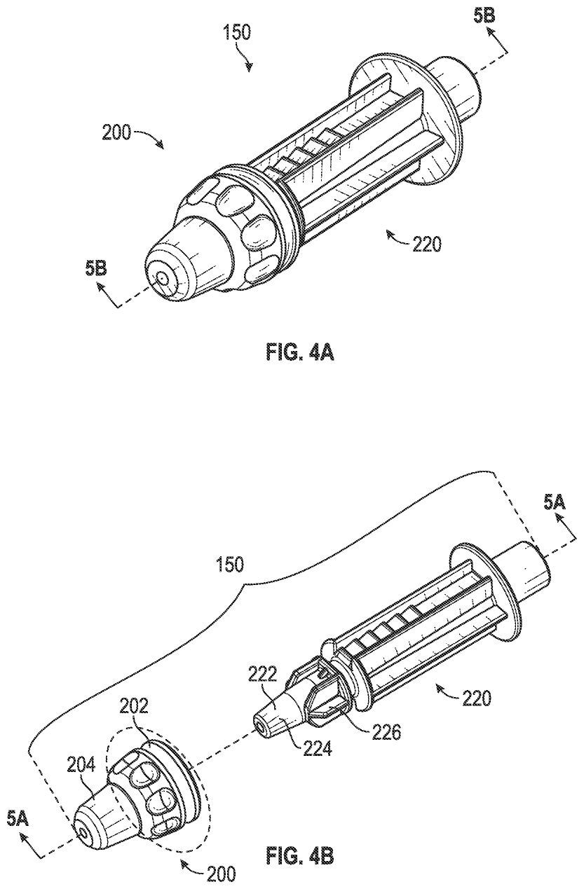

With references to FIGS. 4A-SB, the cover 200 can be removably coupled with head 222 of the body 220. The cover 200 can include the cavity 500 (see FIGS. 5A and 5B) formed inside the tip 204 and the base 202. The shape of the cavity 500 can correspond to the shape of the head 222 of the body 220 such that the head 222 can be inserted within the cavity 500 of the cover 200 without much clearance. This can prevent the head 222 from slipping away from the cover 200 or the cover 200 slipping off from the head 222.

The cavity 500 can include a first portion 550 and a second portion 560. The first portion 550 can be a space circumferentially surrounded by an inner surface of the base 202. The first portion 550 can receive the flaps 226 of the head 222. The second portion 560 can be a space circumferentially surrounded by an inner surface of the tip 204. The second portion 560 can be narrower than the first portion 550. As described herein, the second portion 560 can receive the distal end 224 of the head 222.

The cover 200 can include a lip 524 that is circumferentially formed at a proximal end of the cover 200 and forming a proximal aperture 520 of the cover 200. The lip 524 can extend inwards towards the center of the proximal aperture 520 from the body of the base 202 such that the end of the lip 524 defines the proximal aperture 520. The proximal aperture 520 may be circular or some other desired shape. The lip 524 can include a tapered edge 522 that can facilitate and guide insertion of the head 222 into the cavity 500 of the cover 200. Once the head 222 is inserted into the cavity 500 of the cover 200, a distal edge 526 of the lip 524 (for example, an edge of the lip 524 that faces towards the vial 190 during use) can abut the rear edge 227 of the flaps 226 to prevent the head 222 from slipping out from the cover 200.

The body 220 can include a distal opening 502, a proximal opening 504, and a channel 506 extending and formed between the distal opening 502 and the proximal opening 504. When the cover 200 is placed on the head 222 of the body 220, the distal opening 502 of the body 220 can substantially align with the distal aperture 300 of the cover 200, as shown in FIG. 5B. The gap between the cover 200 and the head 222 can be minimized to prevent any diverted flow into the gap between the cover 200 and the head 222.

In some embodiments, the width of the channel 506 can stay the same or vary along the length of the piston 150. For example, the width of the channel 506 can, as shown in FIGS. 5A and 5B, gradually increase from the distal opening 502 to the proximal opening 504. In another example, the width of the channel can remain substantially the same between the majority of the channel 506 between the distal opening 502 and the stopper 230, and increase significantly at the proximal end 240. The gradual increase of the width of the channel 506 can facilitate flow through the channel 506.

As described herein and shown in FIG. 5A, the rack 228 can include teeth 510. The teeth 510 can be triangular in shape. The teeth 510 can each include a leading edge 512 and a stopping edge 514 that can facilitate and restrict movement of the piston 150 through the cap member 110, respectively.

FIGS. 6A and 6B illustrate various views of the cap member 110 of the vial adaptor 100. The cap member 110 can include a proximal opening 600, a distal opening 602, an arm 610, and the threaded portion 620. The proximal opening 600 can be dimensioned to receive the piston 150. The distal opening 602 can be dimensioned to, for example, extend over at least a portion of the vial 190 (for example, the threaded portion 290 of the vial 190). The threaded portion 620 can be formed on an inner surface of the distal opening 602 and can couple with a corresponding threaded portion (for example, the threaded portion 290) of the vial 190. As described herein, the interaction between the threaded or interlocking portion 620 of cap member 110 and the threaded or interlocking portion 290 of the vial 190 can allow the cap member 110 of the vial adaptor 100 to removably couple with the vial 190.

The arm 610 can include a first portion 614 and a second portion 616. The first portion 614 can include a first end 614A coupled to an inner circumference 604 of the proximal opening 600 and a second end 614B at a predetermined distance away from the first end 614A. As shown in FIG. 6A, the first portion 614 can extend inwardly (for example, in a direction towards the center of the proximal opening 600) from the inner circumference 604 of the proximal opening 600 between the first end 614A and the second end 614B. The second portion 616 can extend distally (for example, in a direction away from the proximal opening 600 and towards the distal opening 602) from the second end 614B of the first portion 614. The second portion 616 can extend substantially orthogonally with respect to the first portion 614. The second portion 616 can include a first end 616A and a second end 616B, where the first end 616A is attached to the second end 614B of the first portion 614. The second end 616B can be a free end and include a detent 612 formed about the second end 616B of the second portion 616. The detent 612 can a curved end that is curved inwards and extending away from the second portion 616, for example, as shown in FIGS. 6A and 6B.

In some embodiments, the cap member 110 can include more than one of the arm 610. For example, as shown in FIGS. 6A and 6B, the cap member 110 can include two arms 610 that are positioned on opposite sides of the inner circumference 604. Alternatively, the two arms 610 can be positioned 90 degrees from one another or any other suitable angles. The detents 612 of the arms 610 can be curved towards each other.

FIGS. 7A-7C illustrate cross-sectional views of the vial adaptor 100, showing different positions of the piston 150 during use. FIG. 7A illustrates relative position of the piston 150 of the vial adaptor 100 with respect to the vial 190 and the cap member 110 prior to actuation (for example, distal translation of the piston 150 towards the vial 190) Prior to actuation, at least a portion of the piston 150 (for example, the cover 200 and the head 222) may be positioned inside the vial 190. Additionally, the outer circumference of the cover 200 of the piston 150 can abut against the inner surface of the vial 190 to provide water-tight seal between the inner surface of the vial 190 and the cover 200. The water-tight seal can prevent or inhibit any flow of fluid stored in the vial 190 being diverted into a space between the cap member 110 and the body 220 of the piston 150. In some embodiments, the detent 612 of the arms 610 of the cap member 110 can abut against a distal-most tooth 610 of the rack 228 prior to actuation of the piston 150.

FIGS. 7B and 7C illustrates relative position of the piston 150 with respect to the vial 190 and the cap member 110 during distal translation of the piston 150. As described herein, the piston 150 (or the body 220 of the piston 150) can be actuated or translated distally towards the vial 190 (for example, as indicated by a directional arrow A). When the piston 150 moves in the direction A, the cover 200 and the head 222 of the piston 150 moves further into the vial 190. This causes a positive pressure change or volume displacement in the vial 190, which causes a flow of fluid stored in the vial 190 out of the vial 190 via the distal aperture 300 of the cover 200, the distal opening 502, the channel 506, and the proximal opening 504 (for example, as indicated by directional arrows B). Additionally, when the piston 150 moves in the direction A (for example, moving distally towards the vial 190), the arm 610 of the cap member 110 can move along the rack 228 towards the stopper 230 by sliding long the leading edges 512 of the teeth 510. The teeth 510 of the rack 228 can be shaped such that the arm 610 can move towards the stopper 230 (for example, by sliding along the leading edges 512) but not towards the head 220. When the piston 150 is pulled in a direction away from the vial 190 (for example, in a direction opposite of direction A as shown in FIG. 7B), the stopping edge 514 of the teeth 510 of the rack 228 can engage the detent 612 of the arm 610 to prevent or inhibit movement of the piston 150 being pulled away from the vial 190. This can prevent or inhibit negative volume displacement (that is, increase in the volume inside the vial 190) and decrease in pressure in the vial 190, which can lead to re-aspiration of, for example, fluid withdrawn from the vial 190.

When the piston 150 is fully actuated (for example, at its distal-most position with respect to the cap member 110), the stopper 230 of the piston 150 can abut against the proximal end 216 of the cap member. Additionally, the detent 612 of the arm 610 may or may not abut against a proximal-most tooth 510 of the rack 228 when the piston 150 is fully actuated.

With reference to FIGS. 8A and 8B, the vial adaptor 100 can be used to transfer, for example, fluid stored in the vial 190 to a receiving device 800. The proximal portion 240 of the piston 150 can be inserted into an opening 802 of the receiving device 800. To prevent accidental leak from the vial 190 via the vial adaptor 100, the vial 190 and the vial adaptor 100 can be oriented to have the vial 190 positioned below the vial adaptor 100 while the proximal portion 240 is being coupled to the opening 802 of the receiving device 800. In some embodiments, the proximal portion 240 and the opening 802 can be coupled via friction fit. In some embodiments, the proximal portion 240 and the opening 802 can have corresponding attachment features (for example, threads) that allow the proximal portion 240 and the opening 802 to be removably secured to each other.

Once the vial 190 and the vial adaptor 100 are coupled to the receiving device 800, the receiving device 800 can be positioned below the vial 190 and the vial adaptor 100. The piston 150 can then be translated towards the cap member 110 by, for example, pushing the vial 190 and the cap member 110 towards the receiving device 800. Pushing the vial 190 and the cap member 110 towards the receiving device 800 can cause the cover 200 of the piston 150 to move towards or into the vial 190 to generate positive pressure or volume displacement in the vial 190. The positive pressure or volume displacement in the vial 190 can cause flow of, for example, fluid stored in the vial 190 to the receiving device 800.

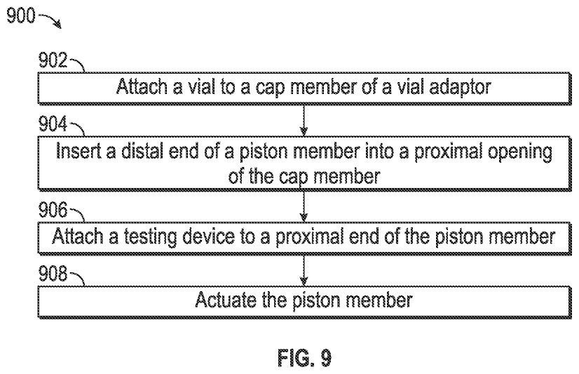

FIG. 9 illustrates an example method 900 of transferring fluid from a vial (for example, the vial 190) to a receiving device (for example, a testing device) using the vial adaptor 100. At step 902, a vial (for example, the vial 190) is attached to the cap member 110 of the vial adaptor 100. For example, as described herein, the vial can include a threaded portion that can be screwed into the threaded portion 620 of the cap member 110. At step 904, the distal end (for example, the cover 200) of the piston 150 is inserted into the cap member 110 via the proximal opening 600 (see FIG. 6A). In some embodiments, the piston 150 is integrated with the cap member 110 while being able to, for example, translate distally towards the vial 190, and as such, the step 904 may be optional.

At step 906, an opening of a receiving device (for example, a testing device or a cartridge) is coupled to the proximal portion 240 of the piston 150. In some embodiments, the opening of the device and the proximal portion 240 can have corresponding coupling features (for example, threads, snap-fit, or a single thread and lock coupling) that allow the piston 150 to be removably secured to the opening of the receiving device. In some embodiments, the proximal portion 240 of the piston 150 can be coupled to the opening of the receiving device via friction fit. At step 908, the piston 150 is actuated. As described herein, the piston 150 may be actuated by distally translating towards the vial 190. When the piston 150 moves towards the vial 190, the movements of the cover 200 and the seal between the cover 200 and the inner surface of the vial 190 can generate positive pressure and volume displacement in the vial 190, which can in turn generate flow out of the vial 190 and into the piston 150. In some embodiments, in order to facilitate the flow out of the vial 190, into the piston 150, and into the receiving device, the vial 190, the vial adaptor 100, and the receiving device (for example, the cartridge 800) may be positioned as shown in FIGS. 8A and 8B.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

The term “comprising” as used herein is synonymous with “including,” “containing,” or “characterized by,” and is inclusive or open-ended and does not exclude additional, unrecited elements or method steps. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A vial adaptor configured to facilitate transfer of fluid from a vial to a receiving device, the vial adaptor comprising:
a cap member comprising a proximal opening, a distal opening, and a threaded portion configured to removably couple with a corresponding threaded portion formed on an opening of a vial;
a piston comprising a cover, a distal end, a proximal end, a channel, and a body extending between the distal end and the proximal end and comprising a head, the cover configured to surround the head and abut an inner surface of the vial, the channel extending between the distal end and the proximal end of the piston, wherein the channel is configured to allow flow of fluid through the piston between the distal end and the proximal end;
a ratchet system comprising:
a first arm formed about the proximal opening of the cap member, at least a portion of the first arm extending inward towards a center of the proximal opening of the cap member, the first arm comprising a tip; and
a first rack formed along the body of the piston, the first rack configured to engage the first arm, wherein the engagement between the first rack and the first arm allows the piston to translate in a first direction;
wherein a translation of the piston in the first direction is configured to cause a positive pressure change in the vial and withdrawal of fluid stored in the vial through the channel of the piston.

2.-48. (canceled)

49. The vial adaptor of claim 1, wherein the first direction is a direction towards the vial.

50. The vial adaptor of claim 1, wherein the engagement between the first rack and the first arm prevents the piston from translating in a second direction.

51. The vial adaptor of claim 50, wherein the second direction is a direction away from the vial.

52. A piston for withdrawing fluid stored in a vial, the piston comprising:
a distal end;
a proximal end;
a body extending between the distal end and the proximal end, the body comprising a head comprising a distal aperture, the proximal end comprising a proximal aperture;
a channel formed within the body and extending between the distal aperture and the proximal aperture;
the head of the piston configured to irreversibly translate towards and into a vial and generate a positive pressure change in the vial, thereby causing a flow of fluid stored in the vial into the channel via the distal aperture and out of the channel via the proximal aperture.

53. The piston of claim 52, wherein a width of the channel increases from the distal aperture to the proximal aperture.

54. The piston of any of claim 52 further comprising a rack comprising a plurality of teeth.

55. The piston of claim 54, wherein the rack is formed along a length of the body of the piston.

56. The piston of claim 54, wherein each of the plurality of teeth comprises a leading edge and a stopping edge.

57. The piston of claim 56, wherein the leading edges of the plurality of teeth are configured to allow an arm of a cap member to slide along in a first direction, and wherein the stopping edges of the plurality of teeth are configured to prevent the arm of the cap member from moving in a second direction opposite from the first direction.

58. The piston of any of claim 52, wherein the piston is configured to move through a proximal opening and a distal opening of a cap member, and wherein the cap member is configured to be attached to an opening of the vial.

59. The piston of claim 58, wherein the cap member is positioned between the piston and the vial.

60. The piston of claim 52 further comprising a cover, the cover configured to cover the distal end of the piston, and wherein an outer circumference of the cover is configured to abut an inner surface of the vial to create a seal between the cover and the vial.

61. The piston of claim 60, wherein the cover is a molded elastomer.

62. The piston of claim 52, wherein the proximal end of the piston is configured to removably couple with a receiving device.

63. A method of withdrawing fluid stored in a vial via a vial adaptor, the method comprising:
coupling a distal opening of a cap member of a vial adaptor to an opening of a vial, the cap member comprising a proximal opening opposite of the distal opening;
coupling a proximal end of a piston of the vial adaptor to an opening of a receiving device;
distally translating the piston towards the vial and through the proximal opening and the distal opening of the cap member, a head of the piston configured to abut an inner surface of the vial to create a water-tight seal between the head of the piston and the inner surface of the vial, a distal translation of the piston towards the vial configured to generate positive pressure change in the vial and cause a flow of fluid stored in the vial, wherein the piston is configured to resist motion in a direction opposite the distal translation.

64. The method of claim 63, wherein the head of the piston is a molded elastomer.

65. The method of claim 63, wherein the cap member comprises an arm and the piston comprises a rack comprising a plurality of teeth, and wherein the arm of the cap member and the plurality of teeth of the piston are configured to engage during the distal translation of the piston.

66. The method of claim 65, wherein each of the plurality of teeth of the rack comprises a leading edge and a stopping edge, wherein the arm slides along the leading edges of the plurality of teeth of the rack during the distal translation of the piston, and wherein the stopping edges of the plurality of teeth of the rack are configured to prevent a proximal translation of the piston away from the vial.

67. The method of claim 63, wherein the piston comprises a channel extending between a distal aperture formed about a distal end of the piston and a proximal aperture formed about the proximal end of the piston.
